(19) United States
(12) Patent Application Publication
(10) Pub. No.: US 2020/0330013 A1
(43) Pub. Date: Oct. 22, 2020

(54) UNIT FOR COLLECTING BLOOD

(71) Applicant: AIM INC., Suwon-si (KR)

(72) Inventors: Yong Ho Cho, Suwon-si (KR); Young Joo Moon, Suwon-si (KR); Woong Ho Lee, Yongin-si (KR)

(21) Appl. No.: 16/854,287

(22) Filed: Apr. 21, 2020

(30) Foreign Application Priority Data
Apr. 22, 2019 (KR) 10-2019-0046655
Jul. 11, 2019 (KR) 10-2019-0083932

(51) Int. Cl. A61B 5/15

(52) U.S. Cl. CPC A61B 5/150419; A61B 5/150358

(57) ABSTRACT

Disclosed herein is a unit for collecting blood including: a handle having a length to be gripped in a hand of a user; and a handling block connected to the handle, wherein the handling block includes: a body disposed to have a second central axis offset from a first central axis of the handle; a collection port formed to pass through the body in a vertical direction and configured to collect blood due to a capillary force; and a discharge groove formed concavely in the body in communication with a lower portion of the collection port to promote discharging of blood absorbed into the collection port to an absorption pad of a diagnostic device when the body is placed on the absorption pad.

### CROSS REFERENCE TO RELATED APPLICATION

This application claims the benefit of Korean Patent Applications No. 10-2019-0046655, filed on Apr. 22, 2019, entitled “Unit for Collecting Blood” and No. 10-2019-0083932, filed on Jul. 11, 2019, entitled “Unit for Collecting Blood”, which are hereby incorporated by reference in their entirety into this application.

### BACKGROUND

### Field

The present invention relates to a unit for collecting blood in a non-invasive form for a simple blood test.

### Description of the Related Art

With the development of medical technology, various kinds of diseases can be diagnosed by blood tests. As a way of collecting blood for a blood test, a syringe is typically used. However, the syringe may cause great pain to a subject from whom blood is collected because the blood is inhaled in a state where a needle of the syringe penetrates into a human body.

Unlike blood collection using a syringe, a non-invasive collection device is also used to collect blood that is taken out after a small wound is made on a finger or the like with a lancet. The non-invasive collection device is used mainly to collect a small amount of blood, and does not cause great pain to a subject from whom blood is collected.

In spite of this advantage, in a conventional cup-shaped blood collector that has a large depth, which is used according to an increase in an amount of blood to be collected, an air layer may be formed inside the cup-shaped structure. Blood collection is disrupted by the air layer, and it is not possible to accurately collect a desired amount of blood.

### SUMMARY

An object of the present invention is to provide a unit for collecting blood using a capillary phenomenon, while not structurally causing a restriction by an air layer in collecting a desired amount of blood.

According to an exemplary embodiment of the present invention, there is provided a unit for collecting blood including: a handle having a length to be gripped in a hand of a user; and a handling block connected to the handle, wherein the handling block includes: a body disposed to have a second central axis offset from a first central axis of the handle; a collection port formed to pass through the body in a vertical direction and configured to collect blood due to a capillary force; and a discharge groove formed concavely in the body in communication with a lower portion of the collection port to promote discharging of blood absorbed into the collection port to an absorption pad of a diagnostic device when the body is placed on the absorption pad.

The body may include: a connection portion to which the handle is connected; and a contact portion located under the connection portion and having a gradually smaller cross-sectional area as being farther away from the connection portion.

The handle may include: a main portion extending along the first central axis; and a sub portion extending along a third central axis that is inclined with respect to the first central axis and intervening for connection between the main portion and the body.

The collection port may be formed to have a gradually smaller cross-sectional area from a lower portion to an upper portion of the body.

The discharge groove may extend radially from the center of the collection port.

According to another exemplary embodiment of the present invention, there is provided a unit for collecting blood including: a handle having a main portion extending along a main axis and formed to be gripped in a hand of a user and a sub portion extending from the main portion to be disposed along a sub axis that is inclined with respect to the main axis; and a handling block connected to the sub portion and disposed along a block axis that is parallel to the main axis, wherein the handling block includes: a body having a connection portion connected to the sub portion and a contact portion located under the connection portion; and a collection port formed to pass through the body in a vertical direction and configured to collect blood due to a capillary force, a width of a lower end of the contact portion being smaller than that of an upper end of the connection portion.

The collection port may be formed to have a gradually smaller cross-sectional area from a lower portion to an upper portion of the body.

The body may further include a discharge groove formed concavely in the body in communication with a lower portion of the collection port to promote discharging of blood absorbed into the collection port to an absorption pad of a diagnostic device when the body is placed on the absorption pad.

According to still another exemplary embodiment of the present invention, there is provided a unit for collecting blood including: a handle extending along a first central axis; and a handling block connected to the handle and disposed to have a second central axis offset from the first central axis, wherein the handling block includes: a body having a connection portion connected to the handle and a contact portion located under the connection portion; and a collection port formed to pass through the body in a direction from the contact portion to the connection portion and configured to collect blood due to a capillary force, the collection port including a section in which a cross section thereof is gradually smaller in the direction from the contact portion to the connection portion.

The contact portion may have a tapered shape such that an outer diameter thereof is gradually reduced as being farther away from the connection portion.

### BRIEF DESCRIPTION OF THE DRAWINGS

FIG. 1 is a front view of a unit 100 for collecting blood according to an exemplary embodiment of the present invention.

FIG. 2 is a cross-sectional view of the unit 100 for collecting blood of FIG. 1.

FIG. 3 is a partially enlarged cross-sectional view of a handling block 150 of FIG. 2.

FIG. 4 is a bottom view of the unit 100 for collecting blood of FIG. 2.

FIG. 5 is a cross-sectional view of a unit 200 for collecting blood according to another exemplary embodiment of the present invention.

FIG. 6 is a bottom view of the unit 200 for collecting blood of FIG. 5.

### DETAILED DESCRIPTION

Hereinafter, a unit for collecting blood according to a preferred exemplary embodiment of the present invention will be described in detail with reference to the accompanying drawings. Throughout the present specification, the same or similar reference numerals denote the same or similar components even in different exemplary embodiments. Once the same or similar components are described, the description thereof will not be repeated.

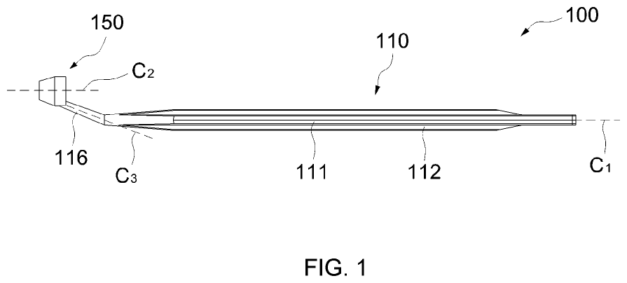

FIG. 1 is a front view of a unit 100 for collecting blood according to an exemplary embodiment of the present invention.

Referring to FIG. 1, the unit 100 for collecting blood may include a handle 110 and a handling block 150.

The handle 110 is a part used to be gripped in a hand of a user. To this end, the handle 110 may have a generally elongated rod shape. Specifically, the handle 110 may include a main portion 111 and a sub portion 116. The main portion 111 may be longer than the sub portion 116. A main axis or first central axis C₁ serving as an extension reference of the main portion 111 may be offset with respect to a block axis or second central axis C₂, which is a central axis of the handling block 150. A rib 112 may protrude on the main portion 111 to prevent the unit 100 for collecting blood from slipping out of the user's hand when the user is gripping the unit 100 for collecting blood. The sub portion 116 is a part connecting the main portion 111 and the handling block 150. When the sub portion 116 extends along a sub axis or third central axis C₃, the third central axis C₃ may have an arrangement relationship with the first central axis C₁ to be inclined with respect to the first central axis C₁.

The handling block 150 is configured to collect blood by being in contact with the blood or discharge the collected blood to an absorption pad of a diagnostic device. The handling block 150 may be connected to the handle 110, in particular an end of the sub portion 116, so that the handling block 150 may be located at one end of the unit 100 for collecting blood.

According to this configuration, the user may handle blood using the handling block 150 while gripping the handle 110, in particular the main portion 111, in the hand.

To do so, the user first draws blood from a finger of a subject to be examined using a lancet or the like. Thereafter, when the handling block 150 is brought into contact with the drawn blood, the handling block 150 collects an appropriate amount of blood due to the action of a capillary force.

Next, the user places the handling block 150 on the absorption pad of the diagnostic device while gripping the handle 110 in the hand. Then, the blood collected in the handling block 150 may be discharged to the absorption pad due to an absorption force of the absorption pad. Thereafter, the user may check a simple diagnosis result for a disease based on the blood through the diagnostic device.

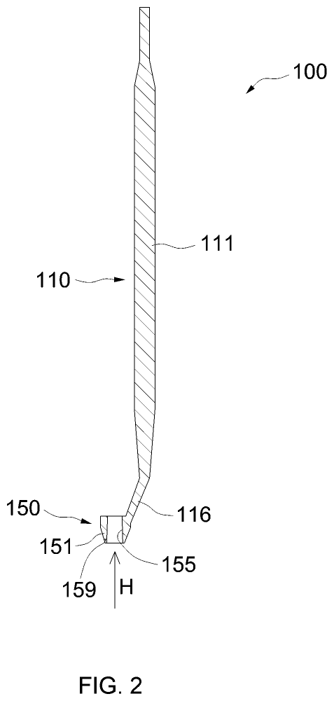

The specific configuration of the above-described unit 100 for collecting blood will be described with reference to FIGS. 2 to 4.

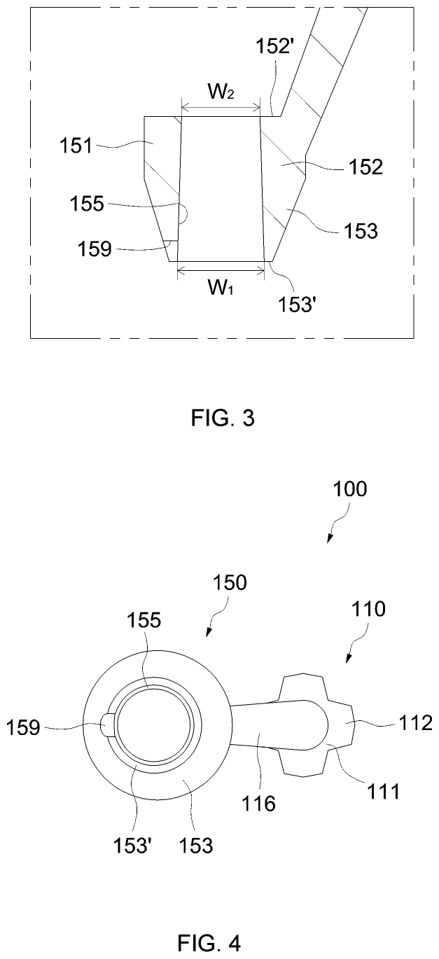

FIG. 2 is a cross-sectional view of the unit 100 for collecting blood of FIG. 1, and FIG. 3 is a partially enlarged cross-sectional view of the handling block 150 of FIG. 2.

Referring to FIGS. 2 and 3, the handling block 150 may include a body 151, a collection port 155, and a discharge groove 159.

The body 151 may have a generally cylindrical shape. Specifically, the body 151 may include a connection portion 152 and a contact portion 153. The connection portion 152 is located in an upper portion of the body 151 and connected to the sub portion 116. The contact portion 153, which is located in a lower portion of the body 151, is a part that is brought into contact with a finger of a subject to be diagnosed or the absorption pad of the diagnostic device.

The contact portion 153 may have a gradually smaller cross-sectional area as being farther away from the connection portion 152. Thus, a width of a lower end 153′ of the contact portion 153 is smaller than that of an upper end 152′ of the connection portion 152. Due to the tapered shape of the contact portion 153, when the contact portion 153 is in contact with a finger from which blood is drawn, it is possible to minimize an amount of blood that is contact with the bottom of the contact portion 153 rather than the collection port 155. As a result, it is possible to accurately adjust an amount of blood collected from the subject to be diagnosed to an amount set by the collection port 155, thereby minimizing a deviation in the amount of blood collected each time.

The collection port 155 is an open groove formed to pass through the body 151 in a vertical direction. Specifically, the collection port 155 may be continuously formed from the lower end 153′ of the contact portion 153 to the upper end 152′ of the connection portion 152. The direction from the lower end 153′ to the upper end 152′ may be defined as a collection direction H.

The collection port 155 may have a gradually smaller cross-sectional area from the contact portion 153 to the connection portion 152 along the collection direction H. Thus, a width W₁ of the lower end 153′ is larger than a width W₂ of the upper end 152′. According to this configuration, when blood is collected due to the action of the capillary force, the collection port 155 has a gradually smaller capillary force as being closer to the upper end 152′ along the collection direction (H). To cope therewith, the width of the collection port 155 is reduced to supplement the capillary force so that the blood may move up to the upper end 152′.

The discharge groove 159 is configured to facilitate the discharging of the blood collected in the collection port 155 to the absorption pad of the diagnostic device. The discharge groove 159 may be formed in the lower end 153′ of the contact portion 153.

The configuration of the discharge groove 159 will be described with reference to FIG. 4.

FIG. 4 is a bottom view of the unit 100 for collecting blood of FIG. 2.

Referring to FIG. 4, the discharge groove 159 is concavely formed in the lower end 153′ of the contact portion 153 in communication with a lower portion of the collection port 155. Specifically, the discharge groove 159 may extend radially from the center of the collection port 155.

The discharge groove 159 structurally increases a cross-sectional area of the lower portion of the collection port 155. Accordingly, it is possible to increase an area in which the blood collected in the collection port 155 is in contact with the absorption pad, thereby promoting the discharging of the blood from the collection port 155 to the absorption pad.

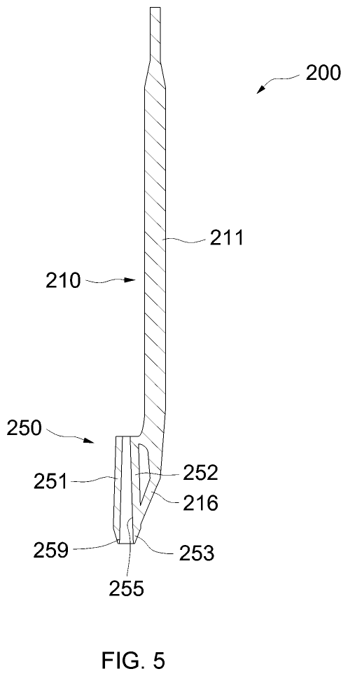

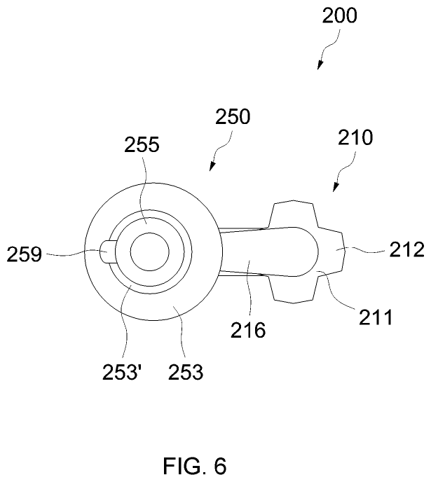

Another form of unit 200 for collecting blood will be described with reference to FIGS. 5 and 6.

FIG. 5 is a cross-sectional view of the unit 200 for collecting blood according to another exemplary embodiment of the present invention, and FIG. 6 is a bottom view of the unit 200 for collecting blood of FIG. 5.

Referring to FIGS. 5 and 6, the unit 200 for collecting blood according to the present exemplary embodiment generally has the same configuration as the unit 100 for collecting blood according to the previous exemplary embodiment. Thus, the unit 200 for collecting blood has a handle 210 and a handling block 250.

The handling block 250 has a height greater than that of the above-described handling block 150, and thereby, a collection port 255 is longer and a larger amount of blood is collected therein. When the collection port 155 in the previous exemplary embodiment is for collecting 10 μl of blood, the collection port 255 in the present exemplary embodiment may be for collecting 20 μl of blood.

Due to an increase in a distance of upward movement of blood, a width of an upper end 252′ relative to that of a lower end 253′ may be smaller when compared to that in the previous exemplary embodiment. According to this configuration, blood rises to a height greater than that in the previous exemplary embodiment, and a larger amount of blood may be collected in the collection port 255.

The blood collected in the collection port 255 may also be smoothly discharged to the absorption pad through the discharge groove 259, while being in contact with the absorption pad in a larger area.

According to the units for collecting blood of the present invention configured as described above, when the handling block is brought into contact with blood in a state where the user is gripping the handle, the collection port formed in the handling block to vertically pass therethrough allows blood to be collected in an amount as much as designed without being disrupted by an air layer, due to a capillary force.

In addition, the discharge groove communicating with the collection port makes it possible to facilitate a process of discharging blood collected in the collection port to the absorption pad of the diagnostic device in a smooth manner.

The above-described units for collecting blood are not limited to the configurations and the operation methods of the exemplary embodiments described above. Each of the above-described exemplary embodiments may also be combined either partially or entirely in a selective manner so that various modifications may be made.

What is claimed is:

1. A unit for collecting blood comprising:
a handle having a length to be gripped in a hand of a user; and
a handling block connected to the handle,
wherein the handling block includes:
a body disposed to have a second central axis offset from a first central axis of the handle;
a collection port formed to pass through the body in a vertical direction and configured to collect blood due to a capillary force; and
a discharge groove formed concavely in the body in communication with a lower portion of the collection port to promote discharging of blood absorbed into the collection port to an absorption pad of a diagnostic device when the body is placed on the absorption pad.

2. The unit for collecting blood of claim 1, wherein the body includes:
a connection portion to which the handle is connected; and
a contact portion located under the connection portion and having a gradually smaller cross-sectional area as being farther away from the connection portion.

3. The unit for collecting blood of claim 1, wherein the handle includes:
a main portion extending along the first central axis; and
a sub portion extending along a third central axis that is inclined with respect to the first central axis and intervening for connection between the main portion and the body.

4. The unit for collecting blood of claim 1, wherein the collection port is formed to have a gradually smaller cross-sectional area from a lower portion to an upper portion of the body.

5. The unit for collecting blood of claim 1, wherein the discharge groove extends radially from the center of the collection port.

6. A unit for collecting blood comprising:
a handle having a main portion extending along a main axis and formed to be gripped in a hand of a user and a sub portion extending from the main portion to be disposed along a sub axis that is inclined with respect to the main axis; and
a handling block connected to the sub portion and disposed along a block axis that is parallel to the main axis,
wherein the handling block includes:
a body having a connection portion connected to the sub portion and a contact portion located under the connection portion; and
a collection port formed to pass through the body in a vertical direction and configured to collect blood due to a capillary force,
a width of a lower end of the contact portion being smaller than that of an upper end of the connection portion.

7. The unit for collecting blood of claim 6, wherein the collection port is formed to have a gradually smaller cross-sectional area from a lower portion to an upper portion of the body.

8. The unit for collecting blood of claim 6, wherein the body further includes a discharge groove formed concavely in the body in communication with a lower portion of the collection port to promote discharging of blood absorbed into the collection port to an absorption pad of a diagnostic device when the body is placed on the absorption pad.

9. A unit for collecting blood comprising:
a handle extending along a first central axis; and
a handling block connected to the handle and disposed to have a second central axis offset from the first central axis,
wherein the handling block includes:
a body having a connection portion connected to the handle and a contact portion located under the connection portion; and
a collection port formed to pass through the body in a direction from the contact portion to the connection portion and configured to collect blood due to a capillary force,
the collection port including a section in which a cross section thereof is gradually smaller in the direction from the contact portion to the connection portion.

10. The unit for collecting blood of claim 9, wherein the contact portion has a tapered shape such that an outer diameter thereof is gradually reduced as being farther away from the connection portion.
